(19) United States
(12) Patent Application Publication
(10) Pub. No.: US 2005/0031653 A1
(43) Pub. Date: Feb. 10, 2005

(54) Sprayable o/w emulsions of a low viscosity

(76) Inventors: Klaus Kwetkat, Bergkamen (DE); Gerd M. Dahms, Duisburg (DE)

Correspondence Address: C James Bushman;Browning Bushman, 5718 Westheimer, Suite 1800, Houston, TX, 77057-5771, US

(21) Appl. No.: 10/487,961

(22) PCT Filed: Aug. 28, 2002
(86) PCT No.: PCT/DE02/03180

(30) Foreign Application Priority Data
Aug. 28, 2001 (DE) 101 41 324.6

(51) Int. Cl. A61K 7/00

(52) U.S. Cl. 424/401

(57) ABSTRACT

This invention relates to sprayable low-viscous O/W emulsions which can be prepared from at least two phases by using a hydrophobic phase comprising gemini surfactants and a hydrophilic phase comprising gemini surfactants with addition of solid particles, a foaming surfactant, or an antitranspirant.

This invention relates to sprayable low-viscous O/W emulsions.

By the term ‘sprayable’ as used herein is meant that the O/W emulsions can directly be sprayed, especially by means of a trigger pump, without using a pressurised propellant.

Sprayable O/W emulsions based on nonionic surfactants and prepared by the phase inversion method or those based on anionic emulsifiers, such as glyceryl stearate citrate or phosphoric acid derivatives, plus a thickener are known in the art.

Sprayable O/W emulsions are very much in demand, particularly in the form of sunscreen products. However, the sprayable O/W emulsions known in the art have the disadvantage that none so far has been capable of taking up the desirable amount of UV-filter combinations which have proved to be tolerable in creams or lotions and comprise oil- or water-soluble organic filters and inorganic ones, e.g. titanium dioxide, which may have an amphiphilic, lipophilic, or hydrophobic coating, and/or zinc oxide. Moreover, it has so far not been possible to use ethanol in higher quantities which would allow to formulate preservative-free products.

In order to produce a particularly fine spray fog and film when using customary spray pumps, it is essential that the compositions to be sprayed have low viscosities. It is also desirable that the hand pump be easy to handle and be presented in an attractive packing design as is expected of cosmetic products. It should produce a fine and steady spray fog, when pumping with steady and moderate energy, irrespective of the speed. Very viscous compositions require solidly built, large-volume, unsightly hand pumps with usually poor spray performance.

Owing to the high densities of inorganic sunscreens in comparison with the aqueous phase, low-viscous O/W emulsions tend to become considerably instable as their pigment concentration increases, i.e. the suspended solid particles tend to agglomerate and/or sediment.

Reportedly, efforts were made to prepare thixotropic pigmentary compositions which can be sprayed under the influence of shearing force, but it has not been possible to prepare sprayable compositions with high solids concentrations.

Solids concentrations of 4% or higher which are typical of suntanning creams or lotions normally present an invincible problem in terms of ‘sprayable composition’. It is hardly feasible to prepare the aforementioned prior-art compositions with higher solids concentrations, especially because the composition when applied to the skin turns to a white layer due to the tendency of the inorganic UV protection particles to agglomerate.

Consumers do not like pump sprays shooting a broad jet onto the skin instead of the fine fog appreciated with aerosol sprays.

Manufacturers of consumer products seek to formulate an emulsifier/dispersant system requiring as few adjustments as possible with the different types of solid particles employed, such as inorganic UV filters, effect pigments for beautifying cosmetics, and fillers for technical applications.

It is, therefore, an object of the present invention to provide a sprayable low-viscous O/W emulsion by means of an adaptable emulsifier/dispersant composition that is easy to use with a multitude of different solids and solids concentrations and which does not have the abovementioned disadvantages of the prior art.

The problem has surprisingly been solved by the following composition:

An oil-in-water (O/W) emulsion having a viscosity of less than 2,000 mPas, preferably 50 to 1,000 mPas, most preferably 500 to 1,000 mPas (at a room temperature of 298 K, measured as dynamic viscosity at a shear rate of 1/s), which can be prepared by combining
(a) a hydrophilic phase comprising
(a.1) one or more gemini surfactant compound(s) and
(a.2) a detergent component with poor foaming characteristics, which is employed at a weight ratio of gemini surfactant compound (a.1) to detergent component (a.2) from 1:100 to 10:1 parts by weight, preferably 1:10 to 4:1, most preferably 1:2 to 2:1, wherein the components (a.1) and (a.2) in total are preferably used in quantities from 0.05 to 5.0 wt %, most preferably 0.5 to 3.0 wt %,
(a.3) 1 to 15 wt % water, most preferably 2 to 10 wt %,
(b) with a hydrophobic phase comprising
(b.1) one or more gemini surfactant compound(s) and
(b.2) one or more co-amphiphile(s) having an HLB value of less than 6, at a weight ratio of gemini surfactant compound (b. 1) to co-amphiphile (b.2) from 1:100 to 3:1 parts by weight, preferably 1:20 to 1:2, most preferably 1:10 to 1:5, wherein the components (b.1) and (b.2) in total are preferably employed in quantities from 0.1 to 8.0 wt %, most preferably 0.5 to 4.0 wt %,
(b.3) 1 to 50 wt % of a hydrophobic component, preferably 10 to 40 wt % and,
(b.4) optionally, 0.01 to 10 wt %, preferably 0.5 to 3 wt % of one or more nonionic surfactant(s),
preferably at elevated temperature, most preferably at 50 to 80° C. or 60 to 70° C., and preferably with homogenisation until the oil droplets have an average particle size of <1 mm, preferably <10 μm,
and additionally comprising
(c.1) water in such quantity that the resultant composition comprises 15 to 45 wt % water, preferably by addition to phase (a) or after combining the phases (a) and (b),
(c.2) alcohols (c.2.1), polyglycols (c.2.2) and/or polyols (c.2.3) such that the whole composition comprises a total of 0.1 to 50 wt % alcohols (c.2.1), preferably 5 to 30 wt %, polyglycols
   (c.2.2) and/or polyols (c.2.3), preferably added to phase (a) and/or (b) and/or after combining the phases (a) and (b), preferably with addition of 0.1 to 10 wt % polyols (c.2.3) to phase (a),
(c.4) optionally, a viscosity regulator preferably in quantities from 0.01 to 3 wt %, more preferably 0.2 to 1 wt %, most preferably added to phase (a) or after combining the phases (a) and (b),
(c.5) optionally, cross-linked polymers, preferably 0.1 to 1 wt %, preferably added after combining the phases (a) and (b),
and adding one or more of the components (d.1) through (d.3) after combining the phases (a) and (b), optionally in conjunction with one or more of the components (c), i.e. (c.1) through (c.5):
(d.1) 0.1 to 30 wt % solid particles, preferably 0.1 to 30 wt %,
(d.2) 0.1 to 3 wt % foaming surfactant, preferably 0.5 to 2 wt %, especially a heavily foaming one, or
(d.3) 0.1 to 15 wt % antitranspirant (antiperspirant), preferably 2 to 8 wt %, for example a metal chlorohydrate, particularly aluminium chlorohydrate or zirconium chlorohydrate,
   with the proviso that when adding the foaming surfactant (d.2), additional water be added, i.e. in addition to the quantity stated in (c.1), such that the proportion of the O/W emulsion comprised of components (a) through (c) to the additional water quantity, including the foaming surfactant, is from 90:10 to 40:60 parts by weight.

It is preferable to add the additional quantity of water conjointly with the foaming surfactant (d.2) or prior to addition of same. After addition of the components specified hereinabove, additional water can be added to the O/W emulsion in larger quantities than those mentioned above.

The O/W emulsion preferably also comprises one or more of the following components:
ethanol in quantities of 0.1 to 20 wt %, preferably 1 to 15 wt %, more preferably 1 to 8 wt %, as component (c.2.1) or part thereof,
(d.4) 0.1 to 25 wt % of a soluble organic sunscreen, preferably added to phase (b), or 0.1 to 25 wt % of a water-soluble sunscreen or the salt thereof added to the water which is added after combining the phases (a) and (b).

It is preferable that (d.2) and (d.3) be added as aqueous solution or emulsion.

The percent by weight specified hereinabove refer to the whole composition of the O/W emulsion. The preferred embodiments of the subject invention are set out in the subordinate claims.

A commercial embodiment comprising the components (a.1) and (a.2) in suitable proportion is the product CERALUTION™ F of Sasol Germany GmbH. A composition containing the components (a.1) and (a.2) is subject matter of WO 01/19943-A1 (PCT/DE 00/03163), which is explicitly incorporated herein by reference.

A particularly useful embodiment containing the components (a.1) and (a.2) consists of sodium lauroyl lactylate and gemini surfactant at a ratio from 1:2 to 2:1 parts by weight.

A commercial embodiment containing the components (b.1) and (b.2) in suitable proportion is the product CERALUTION™ H of Sasol Germany GmbH. A composition containing the components (b.1) and (b.2) is subject matter of WO 01/19945-A1 (PCT/DE 00/03162), which is explicitly incorporated herein by reference.

A particularly useful embodiment with respect to components (b.1) and (b.2) consists of
(1) 10 to 50 wt % behenyl alcohol
(2) 30 to 50 wt % glyceryl stearate
(3) 10 to 20 wt % glyceryl stearate citrate and
(4) 10 to 20 wt % gemini surfactant, preferably of type A I with 15 EO, where ethylene is equal to R², and with acyl residues from a coconut cut (Cl₂, C₁₄, and little C₁₆, the polar terminal group being —SO₃Na).

The gemini surfactants (a.1) and (b.1) can be the same or different.

The O/W emulsions of the invention are outstanding in that >90%, preferably >95%, most preferably >98% of the oil droplets have a diameter of preferably <1 mm, most preferably <10 μm (at least after homogenisation of the phases (a) and (b)). The particle size was determined by a light scattering method using a HORIBA LA-500 particle size analyser.

The hydrophobic phase (b) is defined herein as a composition which is insoluble in water at room temperature, but optionally is water-dispersible. The hydrophilic phase (a) is defined in relation to the respective hydrophobic phase (b). It is preferable that the hydrophilic phase be temporarily water-soluble at room temperature, optionally after previous heating.

The superior spraying characteristics of the O/W emulsions of the invention are demonstrated by the following experiment. A sheet of paper is fixed at a distance of 27 cm from a spray pump head having a round atomizing cone. The paper is sprayed and the sprayed area is measured. The larger the area when using the same hand pump, the better the sprayability of the composition. The compositions of the invention, except the foams containing component (d.2), exhibit excellent spraying behaviour.

The smaller and less disperse the oil droplets, the better an even pigment distribution. The O/W emulsions of the invention allow to distribute pigments in a highly disperse and more even way, thus considerably improving pigment hiding power. This is an advantage in cosmetic applications, e.g. sunscreens, because pigments can be utilised more efficiently and a significantly higher sun protection factor (SPF) per mass unit of solid particle can be attained. In technical applications, too, said emulsions offer advantages, e.g. when it is desirable to keep the solid particles constantly dispersed in their original size without tendency to agglomeration, which is for example advantageous in lacquers.

Adjustment to solid particles which are more hydrophilic can be achieved by increasing the quantities of (a.1) plus (a.2) and, optionally, (b.4) within the abovementioned limits.

(a.1) and (b.1) Gemini Surfactants Compounds

Gemini surfactants can be substantially uniform compounds or mixtures of different compounds.

By the term ‘gemini surfactant’ as used herein is meant a surface-active compound consisting of at least (preferably) two surfactant units, i.e. one hydrophilic head group and one hydrophobic group interlinked through at least (preferably) one spacer in proximity to the head group. Gemini surfactants are also termed dimer surfactants because of their specific structure. There exist anionic, nonionic, cationic, and amphoteric gemini surfactants, depending on the kind of head group. However, in contrast to conventional surfactants, which are grouped in the same way, gemini surfactants can also have combinations of different head groups, mostly combinations of nonionic and ionic groups.

When combining an ionic head group with a nonionic one, it is the nature of the ionic head group which is dominant in the resultant gemini surfactant, combinations of a nonionic head group and an anionic one therefore being classified as anionic gemini surfactants. The same applies to combinations of nonionic head groups with cationic or amphoteric ones. As to the surfactant compositions of the invention, it is morphology (i.e. the relative arrangement of different structural units, namely, hydrophilic groups, spacer, hydrophobic chains) that is essential, the type of head group is not. Hence, the gemini surfactants of the present invention have the following structure:

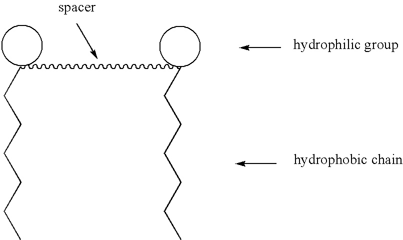

The preferred gemini surfactants used in the surfactant compositions of the invention have nitrogen atoms at the link between spacer, hydrophilic group, and hydro-phobic group. More preferably, the gemini surfactants have spacers bearing amine or amide groups, but also spacers derived from dicarboxylic acids, betaine-derived hydrophilic double head groups, which optionally have side groups obtained by alkoxylation, especially ethoxylation, which may bear sulfonic acid, phosphonic acid, carboxylic acid, or alcohol groups, including polyalcohols, and hydrophobic double chains with 5 to 25 carbon atoms, which may be branched or unbranched and may bear up to two non-adjacent double bonds.

The following variants of gemini surfactant structures are particularly useful for the surfactant compositions of the invention.

Variant A: Structures Based on Amide- or Amine-Containing Spacers
A.I Gemini surfactants of the general formula (A.I) according to WO 96/14926 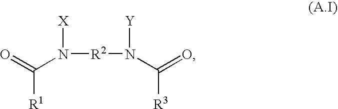
   wherein the substituents independently of one another have the following meanings:
   R¹, R³ C₅- to C₂-5-alkyl, branched or unbranched, saturated, optionally up to two times unsaturated if non-adjacent
   R² C₁- to C₁₂-alkylene
   X, Y (C₂H₄O—)ₓ(C₃H₆O—)_{y}—FR; x+y≧1, x: 0-15, y: 0-10
   FR —SO₃M, —CH₂—CO₂M, —P(O)(OM)₂, H, —C₃H₆SO₃M; or —CH₂(CHOH)₄CH₂OH, insofar as x+y=0, wherein M=a counter-ion, such as alkali, (alkyl)ammonium, alkanol ammonium, H, or ½ alkaline earth
A.II Gemini surfactants having dicarboxylic acid-based spacers of the general formula (A.II) in accord-ance with WO 96/25388
   wherein the substituents independently of one another have the meanings 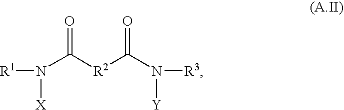
   as defined hereinabove for the general formula (A.I).
A.III Amphoteric gemini surfactants of the general formula (A.III) in accordance with WO 97/31890 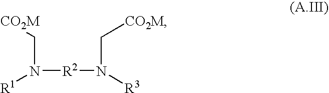
   wherein the substituents independently of one another have the meanings as defined hereinabove for the general formula (A.I). Gemini surfactants of the general formula (A.III) are amphoteric compounds, which can turn into cationic ones if the ambient medium is sufficiently acidic.
      Variant B: Structures Based on Amide- or Amine-Containing Spacers
B.I Gemini surfactants of the general formula (B.I) in accordance with DE 19622612 or
   JP-A 10-175934 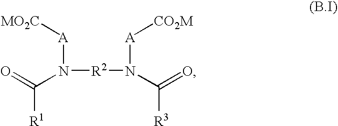
   wherein the substituents independently of one another have the following meanings:
   R¹, R³ C₅- to C₂₅-alkyl, branched or unbranched, saturated, optionally up to two times unsaturated if non-adjacent
   R² C₁- to C₁₂-alkylene
   A CHR⁴, CH₂, C₂H₄, C₃H₆, C₄H₈
   R⁴ aminocarboxylic acid radical
   M a counter-ion, such as alkali, (alkyl) ammonium, alkanol ammonium, H, or ½ alkaline earth.
B.II Gemini surfactants of the general formula (B.II) in accordance with EP 0 708 079 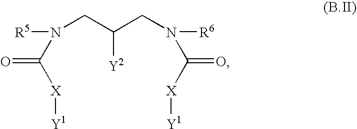
   wherein the substituents independently of one another have the meanings as defined hereinabove for the general formula (B.I) and
   R⁵, R⁶ represent C₆- to C₃₆-alkyl, branched or unbranched, saturated, optionally up to two times unsaturated if non-adjacent;
   X is an alkylene- or alkenylene group having from 1 to 6 carbon atoms, which may be substituted with a hydroxyl group or a sulfonic acid group or a carboxy group;
   Y¹ is a sulfonate- or sulfate group or a carboxyl group
   Y² represents a hydroxyl group, a sulfuric acid residue, or —O—(CO)X—COOH.
B.III Gemini surfactants of the general formula (B.III) according to JP-A-8-311003 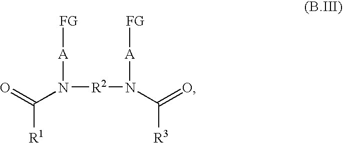
   wherein the substituents independently of one another have the meanings as defined hereinabove for the general formula (B.I) and
   FG represents —COOM or —SO₃M.
B.IV Gemini surfactants of the general formula (B.IV) according to JP-A 11-60437 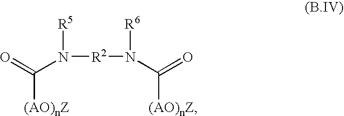
   wherein the substituents independently of one another have the meanings as defined hereinabove for the general formulas (B.I) and (B.II) and
   AO represents alkylene oxide units, i.e. ethyleneglycol-, propyleneglycol-, and butyleneglycol ether units, alone or arranged randomly or blockwise, wherein n=1 to 20, and
   Z is —SO₃M, —C₂H₄SO₃M, —C₃H₆SO₃M, —P(O)(OM)₂ or —CH₂—COOM, —C₂H₄—COOM.
B.V Gemini surfactants of the general formula (B.V) 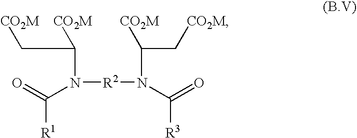
   wherein the substituents independently of one another have the following meaning
R¹, R³ C5- bis C25-alkyl, branched or unbranched, saturated, optionally up to two times unsaturated if non-adjacent;
R² C1- bis C12-alkylene;
M a counter-ion, such as alkali, (alkyl) ammonium, alkanol ammonium, H, or ½ alkaline earth, whereby the carbonic acid groups may as well be only partially neutralized.
   Variant C: Structures Based on Amide- or Amine-Containing Spacers
C.I Gemini surfactants of the general formula (C.I) according to EP 0 697 244, 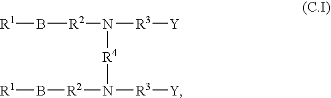
   wherein the substituents independently of one another have the following meanings:
   R¹ C₅- to C₂₅-alkyl, branched or unbranched, saturated, optionally up to two times unsaturated if non-adjacent, hydroxy-substituted or perfluorinated
   R² C₁- to C₁₂-alkylene or hydroxy-substi-tuted derivatives thereof
   B an amide group [—C(O)N(R²)— or
   —N(R⁵)C(O)—], a carboxyl group [—C(O)O— or —OC(O)—], a polyether group
   [—(O(R⁶—O)ₓ—]
   R⁵ C₁- to C₄-alkyl or hydroxy-substituted alkyl or H
   R⁶ C₂- to C₄-alkylene
   x a number from 1 to 20
   R³ C₁- to C₁₂-alkyl or hydroxy-substituted derivatives thereof, R⁷-D-R⁷ or a poly-ether group [—(O(R⁶-O)ₓ—]
   R⁷ C₁- to C₆-alkylene or hydroxy-substi-tuted derivatives thereof
   D —O—, —S—, —N(R⁸)—
   R⁴ alkylene or alkylaryl having from 1 to 12 carbon atoms or the hydroxy-substituted derivatives or R⁹-D¹-R⁹
   R⁸ C₁- to C₁₂-alkyl or hydroxy-substituted alkyl or H or R⁹-D¹-R⁹
   R⁹ C₁- to C₆-alkylene or hydroxy-substituted derivatives thereof or aryl
   D¹ —O—, —S—, —SO₂—, —C(O)—, [—(O(R⁷—O)ₓ—], (R¹⁰)ₜ[N(R¹⁰)]_{z} or aryl
   R¹⁰ C₁- to C₁₂-alkyl or hydroxy-substituted alkyl or H or aryl
   t, z are independently of one another a number from 1 to 4
   Y is independently of one another —SO₃H, O—SO₃H, —OP(O)(OH)₂, —P(O)(OH)₂, —COOH, —CO₂—C₆H₄—SO₃H and the salts thereof.
C.II Gemini surfactants of the general formula (C.II) according to EP 0 697 245 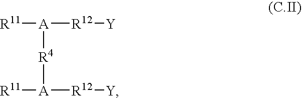
   wherein the substituents independently of one another have the meanings as defined hereinabove for the general formula (C.I) and
   R¹¹ is C₅- to C₂₃-alkyl, branched or un-branched, saturated, optionally up to two times unsaturated if non-adjacent, hydroxy-substituted or perfluorinated or R¹⁴-B-R²,
   R¹⁴ is C₁- to C₁₂-alkyl, branched or un-branched, saturated, optionally up to two times unsaturated if non-adjacent, or the hydroxy-substituted derivatives,
   R¹² means C₁- to C₁₂-alkylene, branched or unbranched, saturated, optionally up to two times unsaturated if non-adjacent, or the hydroxy-substituted derivatives, or an amide group [—C(O)N(R²)— or —N(R⁵)C(O)—], a carboxyl group [—C(O)O— or —OC(O)—], a polyether group [—(O(R⁶—O)ₓ—] or R⁹-D¹-R⁹, and
   A is —CR⁶═ or —N═ with the proviso that if A equals —N═, R¹¹ represents R¹⁴-B-R².
C.III Gemini surfactants of the general formula (C.III) according to DE 4227391 and DE 19608117 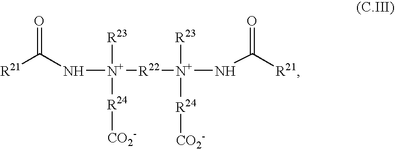
   wherein the substituents independently of one another have the meanings as defined hereinabove for the general formulas (C.I) and (C.II) and
   R²¹ represents C₅- to C₂₃-alkyl, branched or unbranched, saturated, optionally up to two times unsaturated if non-adjacent,
   R²², R²⁴ are C₁- to C₆-alkylene,
   R²³ is methyl, ethyl, propyl, or a polyether group [—(O(R⁶—O)ₓ—].
      Variant D:
D.I Gemini surfactants of the general formula (D.I) according to U.S. Pat. No. 5,863,886 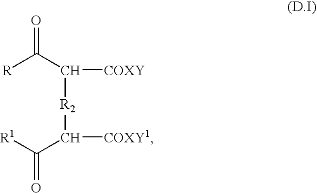
   wherein the substituents independently of one another have the following meanings:
   R, R¹ C₅- to C₃-0-alkyl, branched or unbranched, saturated, optionally up to two times unsaturated if non-adjacent, hydroxy-substituted or perfluorinated
   R² C₁- to C₁₀-alkylene, arylene, and hydroxy-substituted derivatives, a polyether [—O(R⁴O)ₓ—], —S—, —SO₂—, —O—, —S—S—, —O—R⁵—O—, or —S—R⁵—S—
   R⁴ C₂- to C₄-alkylene
   R⁵ C₁- to C₁₀-alkylene, arylene or alkyl arylene, —N(R⁶)—, or —(NR⁶)—R⁷—(NR⁶)—
   R⁶ C₁- to C₆-alkyl
   R⁷ C₁- to C₆-alkyl, wherein R⁷ and R⁶ can also be part of a heterocyclic ring
   X polyether [—O(R⁴O)ₓ—], wherein x is a number from 1 to 30, —O—, NZ
   Z C₁- to C₁₀-alkyl, aryl, alkylaryl, or H, and
   Y, Y₁ are independently of one another H, —CH₂—COOH and salts, a hydrocarbon radical having at least two hydroxyl groups, such as erythrose, threose, ribose, arabinose, xylose, fructose, lyxose, allose, altrose, glucose, mannose, galactose and mixtures thereof.
D.II Gemini surfactants of the general formula (D.II) 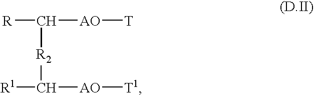
   wherein the substituents independently of one another have the meanings as defined hereinabove for the general formula (D.I) and
   AO means —C(O)—, —C(O)—[—O(R⁴O)ₓ—], —CH₂—[—O(R⁴O)ₓ—], —CH₂—O—,
   T, T¹ are independently of one another —OM, —H, —CH₃, —C₂H₅, —SO₃M, —CH₂COOM, —C₂H₄—COOM, —C₃H₆—SO₃M, —O—P(O)(OM)₂, and
   M is alkyli, ½ alkaline earth, ammonium, mono-, di-, trialkanolammonium, or H.
D.III Gemini surfactants of the general formula (D.III) according to WO 96/16930 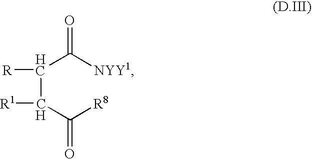
   wherein the substituents independently of one another have the meanings as defined hereinabove for the general formulas (D.I) and (D.II) and
   R⁸ is NYY¹, —O(R⁴O)ₓH, or —O(R⁴O)ₓ—C(O)—CHR—CHR¹—C(O)NYY¹.
D.IV Gemini surfactants of the general formula (D.IV) according to WO 96/25384 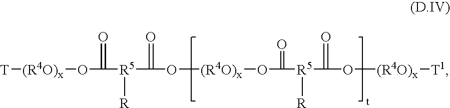
   wherein the substituents have the meanings as defined hereinabove for the general formulas (D.I), (D.II), and (D.III) and
   t is an integer from 1 to 100, preferably 1 to 20, most preferably 1 to 4.

The term ‘up to two times unsaturated if non-adjacent’ employed herein refers to conjugated double bonds.

(a.2) Poor-Foaming Detergent Component

The preferential detergent component characterised by poor foaming and, preferably, mildness is chosen from among the following compounds:

Water-soluble sugar surfactants, acylated protein derivatives, sulphosuccinates, especially sodium- -mono- and -dialkanol sulphosuccinates having branched or unbranched, saturated or mono- to tri-unsaturated if non-adjacent alkyl residues in the range of from C₆ to C₁₈, or acyllactylates, especially sodium-, potassium-, magnesium-, or calcium salts of monomeric lactic acid esterified on the hydroxyl group with linear or branched, saturated or mono- to tri-unsaturated if non-adjacent, cyclic or acyclic C₆- to C₂₄-carboxylic acids, or its oligomers, the oligomerisation degree of the lactic acid being preferably from 1.1 to 10, most preferably from 1.1 to 4, or alkyl(poly)glucosides having an oligomerisation degree of from 1.0 to 10, preferably 1 to 3, and branched

or unbranched, saturated or mono- to tri-unsaturated if non-adjacent, cyclic or acyclic alkyl residues having 6 to 24 carbon atoms, or alkali-, alkaline earth-, mono-, di-, and trialkanolammonium-, ammonium-, mono-, di-, trialkylammonium salts of N-acylated amino acids, optionally also including partially N-acylated oligo-polyamino acids, e.g. alkyl isethionates which comprise alkyl residues with 6 to 24 carbon atoms and are branched or unbranched, saturated or mono- to tri-unsaturated if non-adjacent, or alkali-, alkaline earth-, mono-, di-, and trialkanolammonium-, ammonium-, mono-, di-, trialkyl ammonium salts of acylsarcosinates which comprise alkyl residues with 6 to 24 carbon atoms and are branched or unbranched, saturated or mono- to tri-unsaturated if non-adjacent, or protein condensates having C₆- to C₂₄-acyl residues which are branched or unbranched, saturated or mono- to tri-unsaturated if non-adjacent, or betaines comprising alkyl chains with 6 to 24 carbon atoms, which can be branched or linear, saturated or mono- to tri-unsaturated if non-adjacent. Betaines of the amidoamine type are preferred. Acylglutamates with 6 to 24 carbon atoms in the acyl chain, which can be linear or branched, saturated or mono- to tri-unsaturated if non-adjacent, are also suitable. Particularly preferred detergent components for use in the compositions of the invention are acyllactylates, alkylisethionates and/or acylglutamates or their derivatives.

(b.2) Co-Amphiphiles with HLB Values of Less Than 6

Co-amphiphiles which are solid at room temperature (25° C.) are particularly suitable. Examples of these preferable co-amphiphiles include C₆- to C₄₀-alkyl alcohols, especially C₈- to C₂₄-alkyl alcohols, most preferably cetyl alcohol or behenyl alcohol, which can be branched or unbranched, saturated or mono- to tri-unsaturated if non-adjacent, acyclic or alicyclic, non-neutralized C₆- to C₂-4-alkylcarboxylic acids, preferably C₈- to C₂₂-alkylcarboxylic acids, which can be branched or unbranched, saturated or mono- to tri-unsaturated if non-adjacent, acyclic or alicyclic, alkylaryl derivatives, sorbitan

esters (C₆ to C₂₂), methylglucoside esters (C₆ to C₂₂), sugar esters (C₆ to C₂₂), mono-, di-, and triglycerides of C₆- to C₂₂-carboxylic acids or mixtures thereof, glycerol mono-di-stearate being particularly preferred, branched or unbranched, saturated or mono- to tri-unsaturated if non-adjacent, mono- and di-glycerides of the aforementioned acids and their derivatives which have been further esterified with lactic acid and/or citric acid, C₆- to C₂₂-polyglycerol esters, C₆- to C₂₂-propyleneglycol esters, and also vitamin esters (e.g. vitamin E acetate, vitamin A palmitate), salicylic acid, benzoic acid, lecithins (of vegetable oils or from animals). The alcohols, acids, and mono- and diglycerides of the aforementioned carboxylic acids are especially preferred.

Preferable mixtures are those of long-chain alcohols (C₆- to C₄₀-alcohol, with increasing preference for C₈- to C₂₄-, C₁₄- to C₃₆-, or C₁₄- to C₂₄-alcohol), such as cetyl- or behenyl alcohol, glycerol mono-di-stearate (GMS), and glycerol monostearate esterified with citric acid, or according to another embodiment of the surfactant composition, mixtures of long-chain alcohols, such as cetyl- or behenyl- or erucic alcohol, GMS and stearic acid, most preferably mixtures of behenyl alcohol, GMS, and glycerol monostearate esterified with citric acid.

It is furthermore preferable to use one or more of the following components: Polyethylene glycol derivatives with a large or narrow homologues distribution, polyethylene polypropylene block copolymers, alkyl polyglucose (APG having a DP from 1 to 6), alkyl polyglycerol derivatives, and silicone copolyols.

Preferable gemini surfactant/co-amphiphile(s) compositions independently of one another have besides the gemini surfactant, preferably in quantities from 5 to 25 wt %, most preferably from 10 to 20 wt %, referring to the gemini surfactant/co-amphiphile(s) composition, at least two, preferably three of the different co-amphiphile components as defined hereinafter:
(a) one or more long-chain alcohol(s):
   a C₆- to C₄₀-alcohol, with increasing preference for C₈- to C₂₄—, C₁₄— to C₃₆-, or C₁₄- to C₂₄-al-cohol,
(b) one or more long-chain acid(s):
   a C₆- to C₂₄-carboxylic acid, preferably a C₈- to C₂₂ carboxylic acid,
(c) one or more ester(s)/partial ester(s) of a polyol with one or more mono- or polycarboxylic acid(s):
   a sorbitan(C₆- to C₂₂-)ester,
   a methylglucoside(C₆- to C₂₂-)ester,
   a sugar(C₆- to C₂₂-)ester,
   a mono-, di-, and triglyceride of a C₆- to C₂₂-carboxylic acid,
   a derivative (esterified with lactic acid or citric acid) of the mono- and diglycerides of a C₆- to C₂₂-carboxylic acid,
   a polyglycerol(C₆- to C₂₂-)ester,
   a propyleneglycol(C₆- to C₂₂-)ester,
   a vitamin ester,
(d) and the following additional co-amphiphile components:
   salicylic acid,
   benzoic acid and/or
   lecithin.

According to another embodiment, it is preferable that at least two, more preferable at least three of the components specified hereinbelow be co-amphiphiles:
one or more long-chain alcohol(s) having ≧8 carbon atoms as defined hereinabove under (a) in quantities from 30 to 50 wt %,
a glycerol derivative, e.g. a mono-, di-, and triglyceride of a C₆- to C₂₂-carboxylic acid, or a compound with a similar HLB value, in quantities from 30 to 50 wt %, or
a derivative (esterified with lactic acid or citric acid) of the mono- and diglycerides of a C₆- to C₂₂-carboxylic acid and/or a C₆- to C₂₂-carboxylic acid in quantities from 5 to 25 wt %, preferably 10 to 20 wt %,
   wherein the quantities refer to the gemini surfactant/co-amphiphile(s) composition.

Preferably, the long-chain alcohol is at least one of the co-amphiphiles employed herein, and an ester of a polyol with one or more mono- or polycarboxylic acid(s), preferably having 6 to 22 carbon atoms, is the other (an additional) co-amphiphile.

For example, when using five co-amphiphiles, the composition of the invention preferably contains besides the gemini surfactant the following co-amphiphiles in quan-tities from 5 to 25 wt %, preferably 5 to 20 wt %:
Co-amphiphile 1: long-chain alcohol as defined under (a), in quantities from 20 to 50%, preferably 20 to 35 wt %,
Co-amphiphile 2: an ester/partial ester of a polyol with one or more mono- or polycarboxylic acid(s) as defined under (c), especially GMS or a compound with a comparable HLB value, in quantities from 20 to 50 wt %, preferably 20 to 35 wt %,
Co-amphiphile 3: 5 to 25%, preferably 10 to 20 wt %,
Co-amphiphile 4: 5 to 25%, preferably 10 to 20 wt %, and
Co-amphiphile 5: 5 to 25%, preferably 10 to 20 wt %.

According to a particularly preferable embodiment of the present invention, the composition (a.1+a.2) should be as follows:

| | |
|---|---|
| Gemini surfactant | 5 to 15 wt % |
| Glycerol mono-distearate | 30 to 40 wt % |
| Behenyl alcohol | 35 to 45 wt % and |
| Glycerylstearate citrate | 10 to 20 wt %. |

(b.3) Hydrophobic Component

By the term ‘hydrophobic component’ as used herein is meant mono-, di-, and triglycerides, ester oils, paraffins, cycloalkanes, cyclic and acyclic silicone oils and functionalised silicone oils, long-chain alcohols having chain lengths of >C₁₄, isoprene derivatives, alkyd resins, C₆- to C₄₀-alkyl alcohols, preferably C₈- to C₂₄-alkyl alcohols, most preferably cetyl alcohol or behenyl alcohol, which can be branched or unbranched, saturated or mono- to tri-unsaturated if non-adjacent, acyclic or alicyclic, non-neutralized C₆- to C₂₄-alkylcarboxylic acids, preferably C₈- to C₂₂-alkylcarboxylic acids, which can be branched or unbranched, saturated or mono- to tri-unsaturated if non-adjacent, acyclic or alicyclic, alkylaryl derivatives, sorbitan esters (C₆ to C₂₂), methylglucoside esters (C₆ to C₂₂), sugar esters (C₆ to C₂₂), mono-, di-, and triglycerides of C₆- to C₂₂-carboxylic acids or mixtures thereof, glycerol mono-di-stearate being particularly preferred, branched or unbranched, saturated or mono- to tri-unsaturated if non-adjacent, and mono- and di-glycerides of the aforementioned acids and their derivatives which have been further esterified with lactic acid and/or citric acid, C₆- to C₂₂-polyglycerol esters, C₆- to C₂₂-propyleneglycol esters, and also vitamin esters (e.g. vitamin E acetate, vitamin A palmitate), salicylic acid, benzoic acid, lecithins (of vegetable oils or from animals). The alcohols, acids, and mono- and diglycerides of the aforementioned carboxylic acids, esters of branched and unbranched, saturated and mono- to tri-unsaturated if non-adjacent alcohols with lactic acid, malic acid, salicylic acid, benzoic acid, and tartaric acid.

(b.4) Nonionic Surfactants

The term ‘nonionic surfactants’ employed herein means alkoxylates of branched or linear, saturated or mono- to tri-unsaturated if non-adjacent C₆- to C₂₂-alcohols or alkyl polyglucosides having a polymerization degree of ≧1 or sorbitan ester or sorbitan ester ethoxylates or C₆- to C₂₂-linear or branched alkyl polyglycerides with at least 2 glyceryl units or alkoxylates of mono- and difatty acid glycerides or their mixtures or N,N′-diacylalkylene-diaminealkoxylates or ethylene oxide/propylene oxide block copolymers or N-alkylpyrrolidone derivatives or polyvinyl alcohol derivatives. As used herein, the term ‘alkoxydes’ is defined as ethyleneglycol ethers, propyleneglycol ethers and their combinations arranged blockwise or randomly.

(c.2.1) Alcohols

Preferable alcohols are C₂- to C₄-alcohols, such as ethanol and propanol.

(c.2.2) Polyglycols

Preferable polyglycols are oligomers of polyol compounds which preferably have 2 to 4 carbon atoms and 2 or more hydroxy groups, based on the chain-building monomer. Said oligomers can have as initial or terminal group one or two free hydroxy groups or can have completely etherified initial or terminal groups. C₁- to C₈-mono- or polyols are useful as an initial group, whereas C₁- to C₈-monohydroxy alcohols are suitable as a terminal group. The polyglycols preferably have a molecular weight of not greater than 25,000 g/mol. Examples of suitable polyglycols include polyethylene-, polypropylene-, and polybutylene glycols.

(c.2.3) Polyols

It is preferable that the polyols have 2 to 10 carbon atoms in the (optionally branched) alkylene segment and 2 to 50 hydroxyl groups, most preferably 2 to 6. Suitable polyols include for example alkylene glycols, such as ethylene-, propylene-, butylene-pentylene-, and hexylene glycol and their corresponding isomers, e.g. neopentylglycol. Also suitable are triols, such as glycerol, and higher polyols, such as trimethylolpropane, pentaerythritol, polyglycerol, glucosides, and polyglucosides, saccharides and their corresponding alkyl derivatives and polyvinyl alcohols and mixtures. Also suitable are polyvinyl alcohols.

(c.4) Viscosity Regulators

Suitable viscosity regulators are water—swellable, hydrophilic polymers, such as non-crosslinked polymers, e.g. xanthan gum, linear acrylic acid polymers, pectins, saccharides, cellulose derivatives, caragenan, and arabic gum.

(c.5) Cross-Linked Polymers

Suitable cross-linked polymers are cross-linked polyacrylic acid derivatives and co-polyacrylic acid derivatives, such as copolymerisates with maleic acid.

(d.1) Solid Particles

By the term ‘solid particles’ as used herein is meant particles which are substantially insoluble in the O/W emulsions of the invention. It is preferable that said solid particles substantially have an average particle size of not greater than 20 μm, preferably from 0.01 to 5 μm, referring to the primary particles.

Solid particles can most finely be dispersed and withstand agglomeration by dispersing same, e.g. in a polyol according to (c.2.3), prior to addition to the composition. They may be employed in the form of a paste.

In the O/W emulsions of the invention as much as about 8 wt % titanium dioxide particles can finely be dispersed such that no white layer will appear when spreading the emulsion on the skin. Furthermore, said emulsions are stable. They withstand five freeze-thaw cycles at temperatures ranging from −18° C. to >40° C. and can be stored for two months at room temperature or 50° C. without showing signs of instability.

Suitable solid particles include titanium dioxide with an amphiphilic or hydrophobic coating, zinc oxide, effect pigments which may be coated as well, silica, boron nitrides, carbon black, iron- and aluminium oxides, which may have amphiphilic or hydrophobic coatings, most preferably those coated with silicone derivatives, alumosilicates, aluminas, e.g. bentonites, mica, inorganic pearlescent pigments, metal powder, and metal oxides, such as tin oxide, carmines, ferrocyanides, barium sulfate, bismuth oxychloride, and organic pigments, such as dye pigments and sun protection pigments, e.g. 2,2′-methylene-bis-{6-(2H-benzotriazol-2-yl)-4-(1,1,3,3,-tetramethylbutyl)-phenol}.

A combination of titanium dioxide having an amphiphilic or hydrophobic coating and salicylic ester has proved to be most advantageous. The alcohol group preferably comprises 6 to 22 carbon atoms. The resultant SPF is significantly higher than with a combination of these two components. COSMACOL™ ESI is a useful salicylic ester which is commercially available.

Uses

The sprayable O/W emulsions of the invention can be employed in cosmetics, e.g. as alternatives to skin care creams/lotions or sunscreen lotions which then can have sun protection factors ranging from 2 to 60. Said sprays are also suitable for beautifying cosmetics, e.g. when employing effect pigments, or as liquid/sprayable foundations.

Furthermore, the sprayable O/W emulsions of the invention are useful as leather- or furniture-care products, car-polish auxiliaries, polish auxiliaries required for making silicon wafers, and as top-coat lacquers for (non)absorbent surfaces.

The compositions of the invention are useful as multifunctional preparations for hair/skin cosmetics/detergents, household detergents, furniture-/textile-/carpet-care products and cleaning agents, herbicides, insecticides, and arachnicides.

Foams

For use in emulsion foams produced by means of a trigger pump, not by means of a pressurised agent (propellant) commercialised for example by Airspray International, it is essential that the emulsion has the right viscosity. Since the emulsions in question have exceptionally low viscosities, the same difficulties are encountered as with (sprayable) O/W emulsions with respect to preparation, stability, and versatility. It is not possible to make the emulsions more stable by increasing their viscosity. Since it is difficult to foam ordinary sprayable O/W emulsions and as such emulsions when foamed form poorly stable and not very creamy foams, it was the purpose of the invention to formulate a stable, low-viscous O/W emulsion which can easily be foamed by means of an additional surfactant without losing stability and versatility with respect to the range of oil polarities.

It has surprisingly been found that the problem can be solved by simply adding to a spray prepared from the phases (a) and (b), after it has been made, a 5 to 30% aqueous solution of a heavily foaming surfactant or a preparation containing same. It is preferable that the surfactant added according to the invention be present in quantities from 0.1 to 3.0 wt %, preferably 0.5 to 2.0 wt %, most preferably 1.0 to 2.0 wt %, referring to the O/W emulsion.

Included amongst especially suitable foaming surfactants are alkali-, alkaline earth-, ammonium-, alkanol, and alkylammonium salts of acylglutamates (C₆ to C₂₂, saturated or mono- to tri-unsaturated, branched or linear), alkali-, alkaline earth-, ammonium-, alkanol, and alkylammonium salts of alkyl- or alkenyl isethionates, alkyl- or alkenyl lactates, mixtures of alkyl lactylate salts and one or more of the gemini surfactants specified hereinabove, e.g. CERALUTION™ F, alkylamidopropylbetains, N-acylsarcosinates and other foaming N-acylated amino acids or the salts thereof, and salts of alkyl- or dialkylsulphosuccinates. The foaming surfactant, too, may contain gemini surfactants. The amounts then are employed in addition to the quantities contained in the phases (a) and (b).

Said foams can be prepared by first making the basic O/W emulsion according to the system. The emulsion may be based on a nonionic PIT system or an anionic surfactant, such as glyceryl stearate citrate or a phosphoric ester derivative or gemini surfactant blends. The foaming surfactant then is added, preferably as a solution, at temperatures from 40° C. to 20° C., preferably 35° C.

The mixing ratios of the invention are from 70 to 95 wt % of the low-viscous emulsion, preferably 80 to 95 wt %, and 5 to 30 wt %, preferably 10 to 20 wt % of a 1 to 15 wt % surfactant solution, preferably a 1 to 3% solution.

Experiments

The term ‘mild’ employed herein means that the compounds/compositions are not subject to labelling, e.g. in accordance with EEC guideline 67-548 (Dangerous Substances Ordinance), with regard to their skin and eye irritation potential.

As used herein, the term ‘poor-foaming surfactants’ means that the surfactants when employed as an additional detergent component do not fulfil two requirements of the three specified hereinbelow for evaluating their foaming behaviour, namely
lamellar thickness of the foam (measured in mm) immediately after formation,
number of vesicles in the image detail (with 100 fold magnification) immediately after formation
initial foaming by hand test.

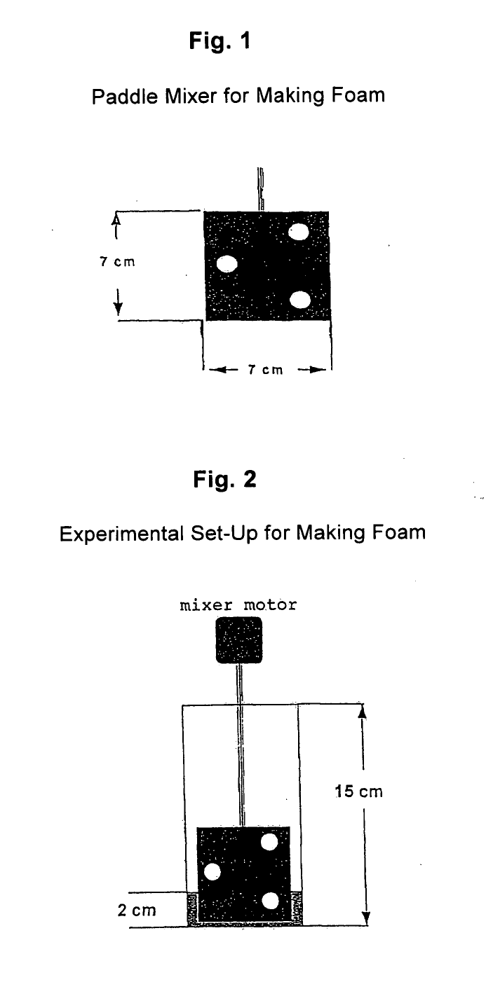

Experiment for evaluating the ‘poor-foaming’ feature:
8 weight percent of the surfactant under examination was dissolved in demineralised water. The surfactant solution was stirred for 10 minutes at 1,500 rpm using the 5 paddle mixer depicted in FIGS. 1/3 through 3/3. The solution was found to heat up slightly from room temperature to approx. 35° C. Stirring was terminated after 10 minutes. The foam produced in this way was skimmed off and promptly examined microscopically. The lamellar thickness was measured in millimetres, and the number of vesicles in the foam was determined in the image detail.

In addition to the foam quality produced by stirring, the initial foaming of a surfactant under running cold tap water was evaluated as well. In this test 2 grams of surfactant were spread over the palms and then evenly rubbed in under running water. The foam quality was evaluated by four grades: 0=no foaming, 1=moderate foaming, 2=good foaming, and 3=very good foaming.

Poor-foaming surfactants are those which do not fulfil at least two requirements of the three specified hereinbelow, namely
a lamellar thickness of less than or equal to 16 mm or
less than or equal to 16 vesicles in the image detail or
grade 1 or less in the initial-foaming test.

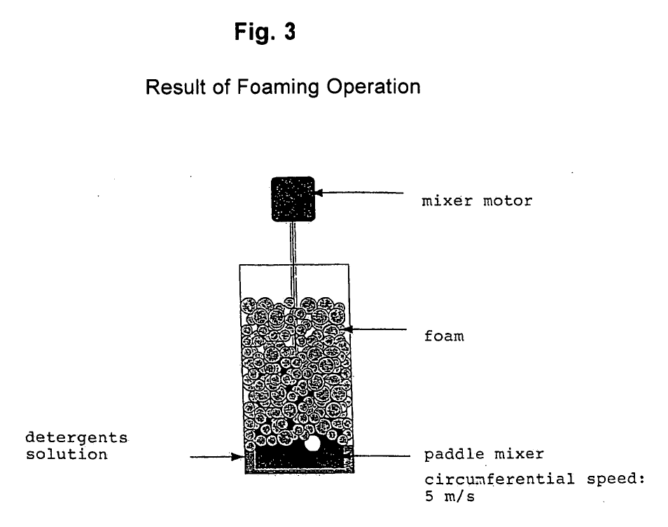

Good-foaming surfactants fulfil all three requirements by the following values: lamellar thickness ≧20 mm, ≧20 vesicles in the image detail (both immediately after initial foaming), and grade 3 initial foaming. The method of making foam is illustrated in FIGS. 1/3 through 3/3. FIG. 1/3 shows the paddle mixer for making foam. FIG. 2/3 represents the experimental set-up for making foam indicating the heights in centimetres, wherein H is the height of the unfoamed solution. FIG. 3/3 shows the foaming result at a circumferential speed of the paddle mixer of 5 m/s. After stirring for 10 minutes, the foam was skimmed off and microscopically examined after 2, 5, and 15 minutes.

Preparation

The sprayable emulsions of the invention are prepared in at least two steps, which will be illustrated by the following example. At 60° C., phase (b) is added to part of the hydrophilic phase (a), which optionally contains the viscosity regulator. Advantageously, the viscosity regulator is a linear polymer, which has previously been swelled in water. The mixture then is homogenised until >95% of the oil droplets, preferably >98% are <1 mm in diameter, preferably <10 μm.

The viscosity is adjusted by increasing or reducing the water quantity in order that the homogenisation period be as short as possible. The product then is allowed to cool to 35 to 40° C., followed by stirring in the residual aqueous phase, optionally with preservative, fragrance, other highly volatile constituents, such as cyclomethicone, and the pigment which has been predispersed in polyol, preferably butyleneglycol. By selection of a suitable component (a.1, a.2; b.1, b.2) it is ensured that the particles will not agglomerate. Surprisingly, the spraying characteristics are not affected by the solid particles content.

The foaming emulsions of the invention are prepared in at least three steps: a sprayable emulsion is prepared as specified hereinabove, a dilute aqueous solution of a foaming surfactant, preferably a heavily foaming one, is admixed, and the product is cooled.

The sprayable antitranspirant emulsions of the invention are prepared in at least three steps: a sprayable emulsion is prepared as specified hereinabove, followed by stirring therein an aqueous solution or suspension or an alkyleneglycol solution or—suspension of metal chlorohydrate, preferably in propylene- or butyleneglycol.

| | | | | | | | | | |
|---|---|---|---|---|---|---|---|---|---|
| Example | | A | | | | | | | |
| Trade Name | CTFA/INCI | [wt.-%] | B | C | D | E | F | G | H |

Phase B or addition to

| | | | | | | | | | |
|---|---|---|---|---|---|---|---|---|---|
| Ceralution H | Behenyl alcohol, glyceryl stearate, | 1.00 | 1.00 | 1.00 | 1.00 | 1.00 | 1.00 | 1.00 | 1.00 |
| | glyceryl stearate citrate, sodium | | | | | | | | |
| | dicocoylethylenediamine PEG-15 | | | | | | | | |
| | sulfate | | | | | | | | |
| Marlipal O13/120 | Isotrideceth-12 | 2.00 | 0.00 | 0.00 | 2.00 | 0.00 | 0.00 | 0.00 | 2.00 |
| Marlipal 1618/25 | Ceteth-25 | 0.00 | 2.00 | 2.00 | 0.00 | 2.00 | 2.00 | 2.00 | 0.00 |
| Miglyol 812 N | Caprylic/capric triglyceride | 25.00 | 25.00 | 25.00 | 25.00 | 25.00 | 5.00 | 14.00 | 25.00 |
| Cosmacol ESI | | | 3.00 | | | | | | |
| | C12-15 Alkyl benzoate | 2.90 | 0.00 | 0.00 | 0.00 | 0.00 | 2.50 | 0.00 | 0.00 |
| | Cyclomethicone | 0.00 | 0.00 | 0.00 | 0.00 | 0.00 | 4.00 | 0.00 | 0.00 |
| | Triethyl citrate | 0.00 | 0.00 | 0.00 | 0.00 | 0.00 | 4.00 | 0.00 | 0.00 |
| | Octocrylene | 0.00 | 0.00 | 0.00 | 0.00 | 0.00 | 5.00 | 4.00 | 0.00 |
| | Octyl methoxycinnamate | 0.00 | 0.00 | 0.00 | 0.00 | 0.00 | 5.00 | 0.00 | 0.00 |
| | Butyl methoxydibenzoylmethane | 0.00 | 0.00 | 0.00 | 0.00 | 0.00 | 1.00 | 0.00 | 0.00 |
| | Tocopherol acetate | 0.00 | 0.00 | 0.00 | 0.00 | 0.00 | 0.50 | 0.00 | 0.00 |
| Uvinul T 150 | Octyl triazone | 0.00 | 0.00 | 0.00 | 0.00 | 0.00 | 0.00 | 4.00 | 0.00 |

Phase A or addition to

| | | | | | | | | | |
|---|---|---|---|---|---|---|---|---|---|
| Demineralised water | | 10.00 | 10.00 | 10.00 | 10.00 | 10.00 | 10.00 | 10.00 | 10.00 |
| Ceralution F | Sodium lauroyl lactylate, sodium | 1.50 | 2.00 | 2.50 | 1.50 | 2.00 | 1.50 | 1.50 | 2.00 |
| | dicocoylethylenediamine PEG-15 | | | | | | | | |
| | sulfate | | | | | | | | |
| Pricerine 9091 | Glycerol | 6.00 | 6.00 | 6.00 | 6.00 | 6.00 | 6.00 | 6.00 | 6.00 |
| Keltrol | Xanthan gum | 0.10 | 0.10 | 0.10 | 0.10 | 0.10 | 0.00 | 0.05 | 0.10 |
| | Hydroxyethylcellulose | 0.00 | 0.00 | 0.00 | 0.00 | 0.00 | 0.20 | 0.00 | 0.00 |

Phase C

| | | | | | | | | | |
|---|---|---|---|---|---|---|---|---|---|
| Demineralised water | Aqua | 34.50 | 29.90 | 27.40 | 33.40 | 27.90 | 25.80 | 28.20 | 32.90 |
| Ethanol | Alcohol | 7.00 | 6.00 | 6.00 | 6.00 | 6.00 | 6.00 | 6.00 | 6.00 |
| | Phenylbenzimidazole-5-sulfonic acid | 0.00 | 0.00 | 0.00 | 0.00 | 0.00 | 4.50 | 4.25 | 0.00 |
| | Sodium hydroxide (10%) | 0.00 | 0.00 | 0.00 | 0.00 | 0.00 | 6.00 | 6.00 | 0.00 |

Phase D

| | | | | | | | | | |
|---|---|---|---|---|---|---|---|---|---|
| ZnO/40% in BG | | 0.00 | 0.00 | 0.00 | 10.00 | 20.00 | 0.00 | 0.00 | 0.00 |
| UV-Sperse T40/BG | | 10.00 | 15.00 | 20.00 | 0.00 | 0.00 | 10.00 | 10.00 | 0.00 |
| | Silica | 0.00 | 0.00 | 0.00 | 0.00 | 0.00 | 0.00 | 0.00 | 10.00 |
| | Butyleneglycol | 0.00 | 0.00 | 0.00 | 0.00 | 0.00 | 0.00 | 0.00 | 2.00 |
| | Pentyleneglycol | 0.00 | 0.00 | 0.00 | 5.00 | 0.00 | 0.00 | 3.00 | 3.00 |
| Total | | 100.00 | 100.00 | 100.00 | 100.00 | 100.00 | 100.00 | 100.00 | 100.00 |
| PSA | | | | | | | | | |

| | | | | | | | | |
|---|---|---|---|---|---|---|---|---|
| median without sonic | 0.32 | 0.34 | 0.37 | 0.34 | 0.35 | 0.34 | 0.36 | 0.33 |
| [μm] | | | | | | | | |
| <1 μm [%] | 98.6 | 99.2 | 98.7 | 99.6 | 98.3 | 98.9 | 99.4 | 98.9 |
| median with sonic [μm] | 0.33 | 0.34 | 0.37 | 0.34 | 0.35 | 0.34 | 0.36 | 0.33 |
| <1 μm [%] | 98.7 | 99.2 | 98.5 | 99.7 | 98.2 | 99.1 | 99.4 | 98.8 |

Preparation:

Heat the phases A and B to 70° C., add phase A to phase B and homogenise until PSA <0.40 μm; cool to 35° C. and admix subsequently phase C and D with homogenisation. BG=butyleneglycol. Gemini: INCI (sodium dicocoylethylenediamine PEG-15 sulfate, cf. structure as defined herein sub A.1, wherein R¹=R³=C₁₁H₂₃—/C₁₃H₂₇, R²=C₂H₄, X=Y=(C₂H₄O—)x(C₃H₆O—)y-SO₃Na, wherein x=15, y=0); Ceralution H (behenyl alcohol: 30-50%, glyceryl stearate: 30-50%, glyceryl stearate citrate: 10-20%, gemini: 10-20%); Ceralution F (sodium lauroyl lactylate: 30-50%, gemini: 30-50%, water: 15%)

| | | | | |
|---|---|---|---|---|
| | | Foaming | | |
| Example | | Emulsion J | Foaming | Sprayable Emulsion |
| Trade Name | CTFA/INCI | [wt.-%] | Emulsion K | with Al-Chlorohydrate L |

Phase B

| | | | | |
|---|---|---|---|---|
| Ceralution H | Behenyl alcohol, | 0.75 | 0.78 | 0.5 |
| | glyceryl stearate, | | | |
| | glyceryl stearate | | | |
| | citrate, sodium | | | |
| | dicocoylethylene- | | | |
| | diamine PEG-15 | | | |
| | sulfate | | | |
| | Steareth-100 | 0.00 | 0.00 | 1.00 |
| Marlipal O13/120 | Isotrideceth-12 | 1.49 | 0.78 | 0.00 |
| Miglyol 812 N | Caprylic/capric | 37.31 | 5.16 | 25.00 |
| | triglyceride | | | |
| | Hydrogenated | 0.00 | 2.35 | 1.05 |
| | cocoglycerides | | | |
| Cosmacol EBI | C₁₂₋₁₅ alkyl | 0.00 | 5.63 | 2.8 |
| | benzoate | | | |
| | Cyclomethicone | 0.00 | 3.91 | 2.5 |
| | Triethyl citrate | 0.00 | 0.00 | 0.00 |
| | Ethylhexylsalicylate | 0.00 | 0.83 | 0.00 |
| | Octyl methoxy- | 0.00 | 10.95 | 0.00 |
| | cinnamate | | | |
| | Benzophenone-3 | 0.00 | 1.56 | 0.00 |
| | Butylmethoxy- | 0.00 | 0.78 | 0.00 |
| | dibenzoylmethane | | | |

Phase A

| | | | | |
|---|---|---|---|---|
| Demineralised H₂O | | 4.26 | 9.32 | 10.00 |
| Ceralution F | Sodium lauroyl | 0.75 | 1.56 | 0.50 |
| | lactylate, sodium | | | |
| | dicocoylethylene- | | | |
| | diamine PEG-15 | | | |
| | sulfate | | | |
| Pricerine 9091 | Glycerol | 8.96 | 7.82 | 6.00 |
| Keltrol | Xanthan gum | 0.37 | 0.31 | 0.15 |

Phase C

| | | | | |
|---|---|---|---|---|
| Demineralised H₂O | Aqua | 32.25 | 35.28 | 34.5 |
| Ethanol | Alcohol | 11.94 | 10.95 | 8.00 |

Phase D

| | | | | |
|---|---|---|---|---|
| | Sodium lauroyl | 1.92 | 2.01 | 0.00 |
| | glutamate* | | | |
| | Sodium | 0.00 | 0.00 | 0.00 |
| | lauroylsarcosinate* | | | |
| | Aluminium | 0.00 | 0.00 | 8.00 |
| | chlorohydrex PG | | | |
| Total | | 100.00 | 100.00 | 100.00 |

| | | | |
|---|---|---|---|
| Additional water required if foaming | 58 | 63 | |
| surfactant is added* | | | |
| Foaming surfactant content | 1.2 | 1.2 | 0.00 |
| (100% active content) | | | |
| PSA | | | |
| median without sonic [μm] | 0.36 | 0.36 | 0.35 |
| <1 μm [%] | 99.4 | 99.2 | 99.5 |
| median with sonic [μm] | 0.36 | 0.34 | 0.34 |
| <1 μm [%] | 99.4 | 99.2 | 99.6 |

Preparation

Heat the phases A and B to 70° C., add phase A to phase B and homogenise until PSA<0.40 μm; cool to 35° C. and admix subsequently phase C and D with homogenisation.
BG=butyleneglycol
PG=propyleneglycol
Gemini: INCI (sodium dicocoylethylenediamine PEG-15 sulfate, cf. structure as defined herein sub A.1, wherein R¹=R³=C₁₁H₂₃-/C₁₃H₂₇, R²═C₂H₄, X=Y=(C₂H₄O—)x(C₃H₆O—)y-SO₃Na, wherein x=15, y=0)
Ceralution H: Behenyl alcohol: 30-50%, glyceryl stearate: 30-50%, glyceryl stearate citrate: 10-20%, gemini: 10-20%)
Ceralution F: Sodium lauroyl lactylate: 30-50%, gemini: 30-50%, water: 15%

### EXAMPLE J

A foaming emulsion can also be prepared by simply blending a sprayable emulsion formulated according to the invention with a dilute surfactant solution, as in this example with 70% sprayable emulsion and 30% of a 10% surfactant solution.

1. (canceled)

2. The O/W emulsion of claim 24, wherein the gemini surfactant compound (a.1) and the detergent component (a.2) with poor foaming characteristics are employed at a weight ratio of gemini surfactant compound (a.1) to detergent component (a.2) from 1:10 to 4:1.

3. An O/W emulsion according to any one of claims 24 or 2, wherein the components (a.1) and (a.2) as a total of gemini surfactant compound (a.1) and detergent component (a.2) are employed in quantities from 0.05 wt % to 5 wt %.

4. An O/W emulsion according to any one of claims 24 or 2, wherein water (a.3) is used in quantities from 2 to 10 wt %.

5. The O/W emulsion of claim 24, wherein the gemini surfactant compound (b.1) and the co-amphiphile (b.2) are employed at a weight ratio of gemini surfactant compound (b.1) to co-amphiphile (b.2) from 1:20 to 1:2.

6. An O/W emulsion according to any one of claims 24, 2 or 5, wherein the components (b.1) and (b.2) as a total of gemini surfactant compound (b.1) and co-amphiphile (b.2) are employed in quantities from 0.1 to 8.0 wt %.

7. An O/W emulsion according to any one of claims 24, 2 or 5, wherein 10 to 40 wt % of the hydrophobic component (b.3) is added.

8. An O/W emulsion according to any one of claims 24, 2 or 5, wherein 0.01 to 10 wt % of one or more nonionic surfactant(s) (b.4), preferably 0.5 to 3 wt % is(are) additionally added to phase (b).

9. An O/W emulsion according to any one of claims 24, 2 or 5, wherein the O/W emulsion comprises 15 to 45 wt % water which is preferably added to phase (a) or after combining the phases (a) and (b).

10. An O/W emulsion according to any one of claims 24, 2 or 5, wherein the O/W emulsion comprises a total of 0.1 to 50 wt % alcohols (c.2.1), polyglycols (c.2.2) and/or polyols (c.2.3), preferably 5 to 30 wt %.

11. An O/W emulsion according to any one of the claims 24, 2 or 5, wherein preferably 0.1 to 10 wt % polyols (c.2.3) are added to phase (a).

12. An O/W emulsion according to any one of claims 24, 2 or 5, wherein the O/W emulsion comprises 0.01 to 3 wt % viscosity regulator (c.4), preferably added to phase (a) or after combining the phases (a) and (b).

13. An O/W emulsion according to any one of claims 24, 2 or 5, wherein the O/W emulsion comprises 0.1 to 1 wt % cross-linked polymers (c.5), preferably added after combining the phases (a) and (b).

14. An O/W emulsion according to any one of claims 24, 2 or 5, wherein the O/W emulsion comprises 1 to 8 wt % solid particles (d.1).

15. An O/W emulsion according to any one of claims 24, 2 or 5, wherein the O/W emulsion comprises 0.5 to 2 wt % foaming surfactant (d.2).

16. An O/W emulsion according to any one of claims 24, 2 or 5, wherein the O/W emulsion comprises a metal chlorohydrate as antitranspirant (d.3), preferably in quantities from 2 to 8 wt %.

17. An O/W emulsion according to any one of claims 24, 2 or 5, wherein the components (d.1), (d.2) or (d.3) are added after combining the phases (a) and (b), optionally in conjunction with one or more of components (c).

18. An O/W emulsion according to any one of claims 24, 2 or 5, wherein the O/W emulsion comprises 0.1 to 20 wt % ethanol, preferably 0.1 to 8 wt %, preferably added to phase (a).

19. An O/W emulsion according to any one of claims 24, 2 or 5, wherein the O/W/emulsion comprises 0.1% to 25 wt % oil-soluble sun protection filter (d.4) added to phase (b) or 0.1% to 25 wt % water-soluble sun protection filter or the salt thereof (d.4) added to the water which is added after combining the phases (a) and (b).

20. An O/W emulsion according to any one of claims 24, 2 or 5, wherein the O/W emulsion comprises as a co-amphiphile (b.2) or the co-amphiphile (b.2) consists of
a C₆ to C₄₀ alcohol, preferably a C₈ to C₂₄ alcohol,
a C₆ to C₂₄ carboxylic acid, preferably a C₈ to C₂₂ carboxylic acid, a sorbitan (C₆- to C₂₂-) ester,
A methylglucoside (C₆- to C₂₂-) ester,
a sugar (C₆- to C₂₂-) ester,
a mono-, di-, and triglyceride of a C₆- to C₂₂-carboxylic acid,
a derivative esterified with lactic acid or citric acid of the mono- and diglycerides of a C₆- to C₂₂-carboxylic acid,
a polyglycerol (C₆- to C₂₂-ester,
a propyleneglycol (C₆- to C₂₂-) ester,
a vitamin ester,
salicylic acid,
benzoic acid and/or
lecithin.

21. An O/W emulsion according to any one of claims 24, 2 or 5, wherein the O/W emulsion comprises as a detergent component (a.2) or the detergent component (a.2) consists of sulphosuccinates, acyllactylates, alkylpolyglucosides, alkylisethionates, acylated protein condensates, betains and/or acylglutamates or the derivatives thereof.

22. An O/W emulsion according to any one of claims 24, 2 or 5, wherein the O/W emulsion comprises as a detergent component (a.2) or the detergent component (a.2) consists of sodium-, potassium-, magnesium- or calcium salts of monomeric lactic acid esterified on the hydroxyl group with linear or branched, saturated or mono- to tri-unsaturated if non-adjacent, cyclic or acyclic C₆- to C₂₄-carboxylic acids, or the oligomers thereof, and wherein the oligomerisation degree of the lactic acid is 1.1 to 10, preferably 1.1 to 4.

23. A process for preparing an O/W emulsion as claimed in any one of claims 24, 2 or 5, wherein the phases (a) and (b) are added to 50 to 80° C., preferably 60 to 70° C., with homogenisation by mixing until the average particle size of the oil droplets is <1 mm as determined by a light scattering method.

24. An oil-in-water emulsion (O/W emulsion) having a viscosity of less than 2,000 mPas, measured at 298 K, which is preparable by combining
(a) a hydrophilic phase comprising
(a.1) one or more gemini surfactant compound(s) and
(a.2) a detergent component with poor foaming characteristics, at a ratio of gemini surfactant compound (a.1) to detergent component (a.2) from 1:100 to 10:1 and
(a.3) 1 to 15 wt % water,
(b) with a hydrophobic phase comprising
(b.1) one or more gemini surfactant compound(s) and
(b.2) one or more co-amphiphile(s) having an HLB value of less than 6, at a weight ratio of gemini surfactant compound (b.1) to co-amphiphile (b.2) from 1:100 to 3:1 parts by weight,
(b.3) 1 to 65 wt % of a hydrophobic component, and furthermore comprising
(c. 1) water in such quantity that the resultant whole composition comprises 15 to 45 wt % water,
(c.2) alcohols (c.2.1), polyglycols (c.2.2) and/or polyols (c.2.3) such that the whole composition comprises a total of 0.1 to 50 wt % alcohols (c.2.1), polyglycols (c.2.2) and/or polyols (c.2.3),
and one or more of the components (d.1) through (d.3) added after combining the phases (a) and (b), optionally in conjunction with one or more of the components(c),
(d.1) 0.1 to 30 wt % solid particles,
(d.2) 0.1 to 3 wt % foaming surfactant or
(d.3) 0.1 to 15 wt % antitranspirant,
with the proviso that when adding the foaming surfactant (d.2), additional water be added such that the proportion of the O/W emulsion comprised of components (a) through (c) to the additional water quantity including the foaming surfactant (d.2), is from 90:10 to 40:60 parts by weight and wherein the gemini surfactants comprise at least two surfactant units, comprising each at least one hydrophilic head group and at least one hydrophobic group, and the surfactant units are interlinked through at least one spacer in proximity to the head group.
